(19) United States
(12) Patent Application Publication
(10) Pub. No.: US 2026/0077110 A1
(43) Pub. Date: Mar. 19, 2026

(54) METHOD, SYSTEM AND APPARATUS FOR REMOVING GAS BUBBLES IN A FLUID CIRCUIT

(71) Applicant: Fenwal, Inc., Lake Zurich, IL (US)

(72) Inventors: Haley Olson, Chicago, IL (US); Johnwesly Kanagaraj, Naperville, IL (US); Zachary DeSalvo, McHenry, IL (US)

(21) Appl. No.: 19/332,792

(22) Filed: Sep. 18, 2025

Related U.S. Application Data
(60) Provisional application No. 63/696,444, filed on Sep. 19, 2024.

(51) Int. Cl. A61M 1/36

(52) U.S. Cl. CPC A61M 1/3627; A61M 1/36226

(57) ABSTRACT

Receptacles for use in fluid lines of aliquot fluid circuit in a fluid distribution system and method of treating air bubbles in fluid lines of an aliquot fluid circuit in a fluid distribution system are disclosed therein. The receptacle is configured to trap and remove gas (e.g., air bubbles) that flows into the receptacle from the fluid lines. The receptacle is in fluid communication with a product (aliquot) container.

### CROSS-REFERENCE TO RELATED APPLICATIONS

This application claims the benefit of and priority to U.S. Provisional Patent Application Ser. No. 63/696,444, filed Sep. 19, 2024, the disclosure of which is incorporated herein by reference.

### TECHNICAL FIELD

The present disclosure relates to controlling the transfer of fluid and, more particularly, to restricting and removing air bubbles in fluid lines of a fluid circuit.

### BACKGROUND

Some blood products and cell therapy applications require products aliquoted into ready to use bioprocess single use bags or containers to eliminate on-site aliquoting workflows and ensure aseptic integrity of the products. Removing air bubbles from product preparations allows for accurate dosing and prevents potential damage to cells and/or tissue. During processing of biological fluid, clinical personnel are often required to manually remove foam and air bubbles contained inside the bags or containers by massaging the collection or product bag or tilting the container and gently pulling out and/or pushing in air to encourage bubbles to rise and coalesce near the port of the container. This manual approach can be difficult and time consuming, which may result in undesirable risk of increasing the DMSO (Dimethyl Sulfoxide) exposure time and impact product integrity. Moreover, several applications may require high accuracy in the volume of fluid transferred between the containers. For example, there are applications that require an accuracy of 0.5 ml or less.

Thus, it would be desirable to provide a system, device and method that address the challenges related to fluid transfer distribution systems and the presence of gas (e.g., air bubbles) in the fluid lines of the fluid transfer distribution system.

### SUMMARY

In an aspect, an aliquot fluid circuit within a fluid distribution system having a distribution pump is provided. The aliquot circuit includes at least one fluid line wherein a first fluid line of the at least one fluid line is fluidly connected to the distribution pump. The fluid circuit includes at least one or more receptacles, wherein each of the at least one or more receptacles includes a housing defining a flow passage, an inlet configured in fluid communication with the distribution pump and an outlet. The flow passage is configured for fluid communication between the inlet and outlet and allows the fluid to flow therethrough and for being collected therein; The at least one or more receptacles are configured to inhibit a flow of gas from the inlet to the outlet.

In another aspect, a method for treating gas bubbles in an aliquot fluid circuit upon filling fluid to the at least one or more product (aliquot) containers. The method includes (i) placing a receptacle above the product container; (ii) connecting the receptacle in line with at least one fluid line, wherein the flow passage is configured for fluid communication with the at least one fluid line; (iii) orienting the receptacle in a default position in which the inlet faces upwardly and the outlet faces downwardly relative to the ground. The method includes (iv) activating the distribution pump to push the fluid to the receptacle, and fill the product container with the fluid. The method further includes (v) activating the distribution pump in a first or second operation mode to draw vacuum from the fluid circuit to divert the gas bubbles residing in the fluid contained in the product container towards the receptacle; (vi) tapping the receptacle to direct the gas bubbles towards the inlet direction to a trapping zone above the fluid residing in the flow passage; (vii) activating the distribution pump in a third operation mode to push the fluid in the receptacle into the product container; and/or (viii) activating the distribution pump in a fourth operation mode to push the fluid in the receptacle into the product container.

In yet another aspect, a method for treating gas bubbles in the aliquot fluid circuit upon filling fluid to the at least one or more product containers, the method comprising steps: (i) placing the receptacle above the product container; (ii) connecting the receptacle in line with the at least one fluid line, wherein the flow passage of the receptacle configured in fluid communication with the at least one fluid line; (iii) orienting the receptacle in an inverted position in which the inlet faces downwardly and the outlet faces upwardly relative to the ground; (iv) activating the distribution pump to push the fluid to fill the receptacle, prior to entering the product container, wherein the gas bubbles float above the fluid residing in the receptacle towards the outlet direction; (v) orienting the receptacle in the default position in which the inlet facing upward and the outlet facing downward relative to the ground to inhibit the gas bubbles flow from the inlet to the outlet without obstructing the fluid flow; (vi) tapping the receptacle to direct the gas bubbles towards the inlet direction to trapping zone above the fluid residing in the flow passage; (vii) activating the distribution pump with a third operation mode to push the fluid in the receptacle into the product container; or (viii) activating the distribution pump with a fourth operation mode to push the fluid in the receptacle into the product container.

### DESCRIPTION OF THE DRAWINGS

FIG. 1A is a perspective view of an aliquot fluid circuit and the associated fluid distribution system.

FIG. 1B is an exemplary aliquot fluid circuit.

FIG. 1C is a perspective view of a fluid device configured in line with an aliquot fluid line for managing air containment in aliquoting containers.

FIG. 2A is an example of Fast Pull Air Out operation mode symbol.

FIG. 2B is an example of Slow Pull Air Out operation mode symbol.

FIG. 2C is an example of Fast Push Air In operation mode symbol.

FIG. 2D is an example of Slow Push Air In operation mode symbol.

FIG. 3A illustrates an exemplary step of the method for treating/removing gas bubbles in the exemplary aliquot fluid circuit.

FIG. 3B illustrates another exemplary step of the method for treating/removing gas bubbles in the exemplary aliquot fluid circuit.

FIG. 3C illustrates a further exemplary step of the method for treating/removing gas bubbles in the exemplary aliquot fluid circuit.

FIG. 3D illustrates a still further exemplary step of the method for treating/removing gas bubbles in the exemplary aliquot fluid circuit.

FIG. 4 is a block diagram of an embodiment of the processing hardware of the fluid processing system 100 of FIG. 1.

### DETAILED DESCRIPTION

A more detailed description of the systems and methods in accordance with the present disclosure is set forth below. It should be understood that the description below of specific devices and methods is intended to be exemplary, and not exhaustive of all possible variations or applications. Thus, the scope of the disclosure is not intended to be limiting and should be understood to encompass variations or embodiments that would be apparent to persons of ordinary skill.

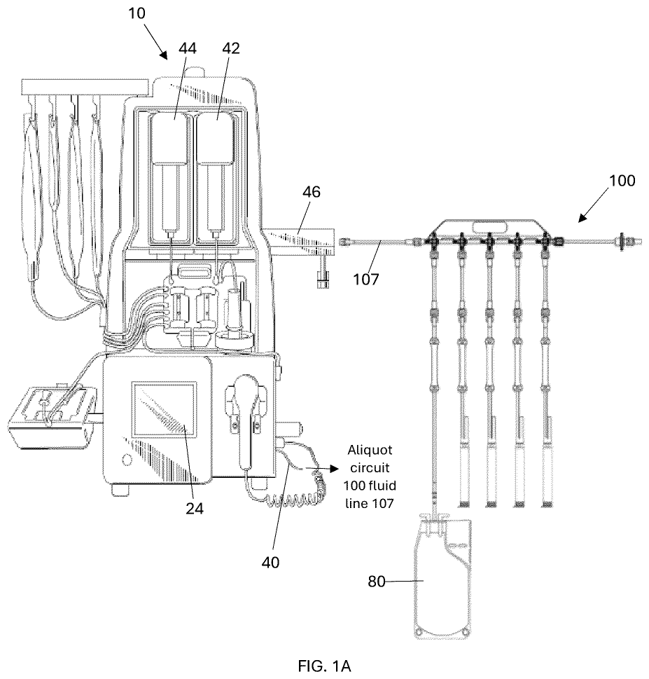

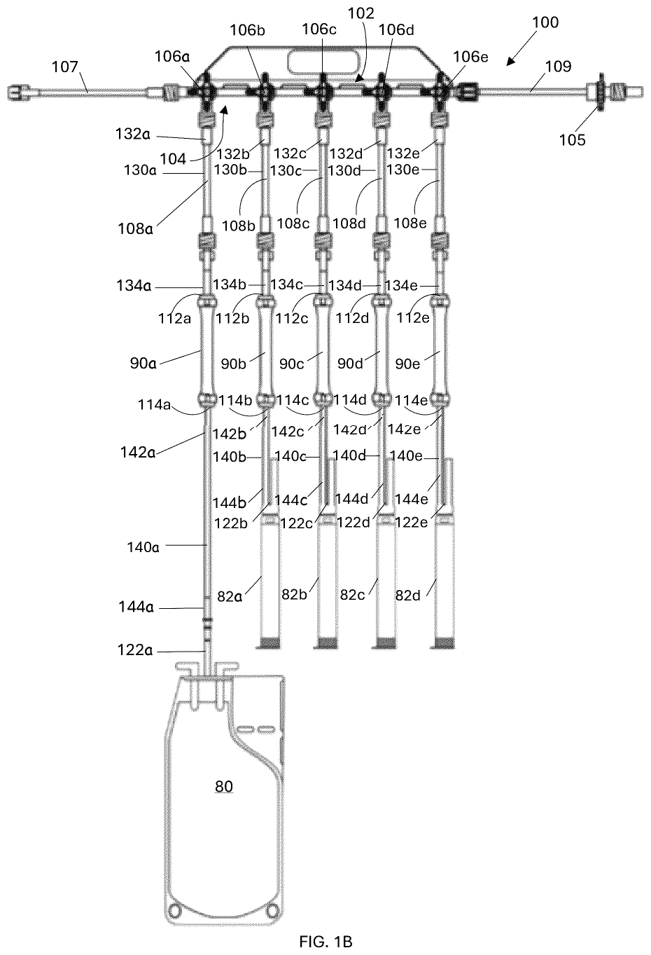

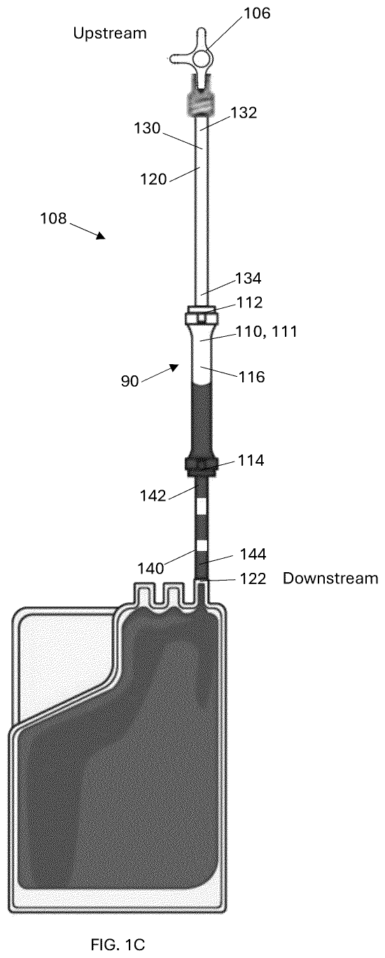

Shown in FIG. 1A is an embodiment of a fluid circuit 100 connected with a fluid distribution system 10 having at least one distribution pump 42, 44. In one embodiment, fluid distribution system 10 may be the Cue Cell Processing System from Fresenius Kabi of Lake Zurich, Illinois.

Turning to FIG. 1B, an embodiment of a fluid circuit 100 at the aliquot stage for distributing fluids from a fluid distribution system 10 to a plurality of product containers 80, 82a-d is illustrated. The circuit 100 may be used to distribute fluid from (i) one container to another, (ii) from several containers to one container, or (iii) from one container to several containers. Furthermore, the circuit may distribute volumes of fluid between the containers at an accuracy of 5 ml or less. Additionally, the circuit 100 is associated with a substantially closed fluid distribution system 10 in that the containers 80, 82a-d may be in fluid communication with each other via substantially closed circuit lines 102.

The circuit 100 has fluid circuit lines 102 associated with a manifold 104 (that may be mounted onto system 10) having a plurality of stopcocks 106a-e and fluid lines 107, 108a-e, 109. These components of the circuit lines 102 may be fluidly connected. The circuit 100 also includes the plurality of product containers 80, 82a-d that are connectable to the aliquot circuit lines 102 via respective fluid lines 108a-e and connectors. The circuit 100 is associated with the fluid distribution system 10 having a distribution pump 42 for transferring/distributing fluid from the fluid distribution system 10 to the product/aliquot containers.

As illustrated in FIG. 1B, circuit 100 further includes a plurality of receptacles (pouches) 90a-e configured in line with the fluid lines 108a-e. The illustrated aliquot circuit 100 includes five receptacles (pouches) 90a-e, but the circuit 100 may include more or fewer than five containers, associated with the number of product/aliquot containers 80, 82a-d, depending on the application.

Each of the receptacles (pouches) 90a-e respectively separates each of the fluid lines 108a-e into two sub-lines which provide a first conduit 130a-e and a second conduit 140a-e. Each of the first conduits 130a-e includes a respective first upstream end 132a-e connected to the respective stopcock 106a-e, and a respective first downstream end 134a-e connected to respective inlet 112a-e of the receptacles 90a-e. Each of the second conduits 140a-e includes a respective second upstream end 142a-e connected to a respective outlet 114a-e of the receptacle (pouches) 90a-e, and a respective second downstream end 144a-e. Optionally the respective second downstream end 144a-e may be connected to the respective inlet port 122a-e of the aliquot (product) containers 80, 82a-d at the time of use.

The first upstream ends 132a-e of fluid line 108a-e are connected to and in fluid communication with manifold 104. The manifold includes at least one stopcock 106a-e for controlling the fluid flow in the circuit 100. The manifold 104 also includes first section 107 of the main aliquot line and second section 109 of main aliquot line. As illustrated in FIG. 1A, the manifold 104, along with the fluid lines 108a-e and stopcocks 106a-e, can be configured to attach to the fluid distribution system 10. This configuration ensures that the manifold has sufficient support, which can be facilitated by a part extended from system 10, such as arm 46 as illustrated, allowing unobstructed fluid flow from the fluid distribution system 10 to the aliquot circuit 102. Still as illustrated, the first section 107 of main aliquot line fluidly connects to port 40 of distribution pump 42 and the second section 109 of main aliquot line fluidly connects to an air filter 105. The one or more stopcocks 106a-e are manipulatable, and the position of each stopcock is controlled manually. The first upstream ends 132a-e of fluid line 108a-e are connected to and in fluid communication with stopcocks 106a-e. In one alternative embodiment, ends 132a-e of fluid lines 108a-e are releasably attached to a respective stopcock 106a-e by, for example, luer fittings.

The illustrated aliquot circuit 100 includes five product/aliquot containers 80, 82a-d, but the circuit 100 may include more or fewer than five containers, depending on the application. The product containers may be welded containers, such as flexible bags made from welded plastic films. In other embodiments, the product containers may be vials, jars, or any other suitable container. In one alternative as illustrated, the product/aliquot containers 80, 82a-d may be a mix of different types of containers, wherein the type of each container depends on the particular application or the type of fluid in the container.

The set of containers 82 may include container 82a, 82b, 82c and 82d. In one configuration of circuit 100, containers 80, 82a-d are each product or aliquot containers which receives fluid distributed from the fluid distribution system 10. Each of the containers 80, 82a-d contain fluid (such as a solution or suspension) that is dosed from the respective supply container associated with the fluid distribution system 10. In one embodiment, the product or aliquot containers 80, 82a-d may contain a blood product (such as whole, blood, plasma, etc.) or cell solution (such as a cell culture).

Each of the product or aliquot containers 80, 82a-d are connected to and in fluid communication with the aliquot circuit lines 102. Containers 80, 82a-d are connected to the second downstream ends 144a-e of respective fluid lines 108a-d. In one embodiment, the containers are releasably connected to the fluid lines via luer locking mechanism. For example, the containers 80, 82a-d each have a port that includes a female or male luer lock, which complementarily engages the corresponding male or female luer lock at the ends 144a-e of fluid lines 108a-e. As exemplified, after a container(s) has/have received the selected dosing, the container(s) may be disconnected, and a new container(s) or, more preferably, a new manifold with new fluid line tubing(s) and containers may be mounted onto system 10 to receive another dosing. Containers connected via luer lock offer flexibility in fluid dosing for various applications. The luer lock mechanism allows for easy and secure attachment and detachment of containers, enabling quick changes and adjustments and having flexibility of different container connections, in addition to sterile welding of containers onto the tubing line. This makes the fluid processing simple to attach/connect containers for different fluids or doses as needed, enhancing versatility and efficiency in fluid management.

Turning now to FIG. 1C, a configuration of a specific circuit line 108a (hereinafter denoted as 108 to generalize the fluid line 108a-e) and its peripheral components mentioned herein is shown. The same configuration may apply to other circuit lines 108b-e in FIG. 1B. The configuration is designed for managing and trapping gas (e.g., air) bubbles dissolved in the fluid distributed within circuit 100 from a fluid distribution system 10 upon filling the aliquoting container 80. Specifically, the circuit line 108 provides the receptacle 90, which when at least partially filled with fluid, traps gas bubbles from the fluid circuit line 108. The receptacle 90 comprises a housing 110 defining a flow passage 116, an inlet 112 at one end of the housing 110 and an outlet 114 at another end of the housing 110 opposite to the inlet 112. The flow passage 116 may be configured in fluid communication with one of the circuit lines 108. For example, the receptacle 90 mechanically connects to the circuit line 108. As shown, the flow passage 116 is configured in fluid communication with the inlet 112 and outlet 114 to allow the fluid flowing therethrough and being collected therein.

The inlet 112 is connected to the first downstream end 134 of the first conduit 130 of circuit line 108. The outlet 114 is connected to the second upstream end 142 of the second conduit 140 of circuit line 108. The inlet 112 and outlet 114 may be releasably coupled to or integrally formed with the first downstream end 134 and second upstream end 142 respectively. The inlet 112 is configured in fluid communication with the stopcock 106 and the outlet 114 is configured in fluid communication with the aliquot product container 80. The flow passage 116, between the inlet 112 and outlet 114, forms a chamber within the housing 110 that allows fluid to flow therethrough and to be collected therein.

As fluid flows through and fills the receptacle or pouch 90, flow passage 116 allows the fluid to flow while creating an air trapping zone 111. In this zone, gas bubbles are diverted from the main flow path, effectively separating them from the fluid. The air trapping zone 111 is located above the fluid in the flow passage 116 when the pouch 90 is substantially filled with fluid. The tubing of circuit line 108 provides a channel 120 for the fluid to be delivered from the fluid distribution system to the aliquoting/product container 80. The receptacle (pouch) 90 is integrated in line with the circuit line 108 and thereby the flow passage 116 of the receptacle 90 is configured in fluid communication with the channel 120 of the circuit line 108. With the receptacle (pouch) 90 being integrated in line with the circuit line 108, the pouch 90 separates the circuit line 108 into two sub-lines which provide a first conduit 130 and a second conduit 140. The first conduit 130 includes a first upstream end 132 connecting to the stopcock 106, and a first downstream end 134 connecting to an inlet 112 of the receptacle 90. The second conduit 140 having a second upstream end 142 connecting to an outlet 114 of the receptacle 90, and a second downstream end 144 connecting to an inlet port 122 of the aliquot container 80. The receptacle 90 may be releasably connected to the circuit line 108 with for example, luer fittings. In an alternative embodiment the fluid device may be integral with the circuit line 108.

The configuration as discussed herein provides a mechanism for trapping and removing gas bubbles which is described below. The circuit line within the fluid distribution system 10 uses the flow dynamics within the tubing and receptacle (pouch) 90 to direct gas/air bubbles into a trapping zone. The fluid flow within the trapping zone may be designed so that bubbles naturally migrate towards an area of lower pressure, while the main fluid stream continues unobstructed. The structural characteristic may involve having the receptacle (pouch) 90 configured to create a region where the fluidic velocity changes, assisting to pull bubbles out of the main flow path to the designated trapping zone. By manipulating the pumping within the fluid distribution system 10, such as applying a positive and/or negative pressure to the aliquot line 102 within the fluidic circuit 100, essentially the manipulation changes the fluid flow velocity. That said, manipulation of distribution pump 42 causes a changing of direction, of applying pressure to the fluid line 108 (i.e., push air in or pull air out) and diverts the gas/air bubbles towards the receptacle 90. The pressure helps push air in or pull air out from the receptacle 90 where the gas in the form of air bubbles can rise to the top of the fluid collected in the receptacle 90 and be removed.

According to the present disclosure, the pump 42 may provide a negative pressure (i.e., vacuum) to draw air bubbles out and provide a positive pressure to push the resultant air bubble-free fluid to the destination container. Such mechanism utilizes flow dynamics within the flow passage 116 to separate air bubbles from the liquid by allowing bubble migration above the fluid residing in the flow passage, where they can be trapped and removed, without obstructing the main fluid stream.

The circuit 100 is associated with the fluid distribution system 10 that includes a distribution pump 42 that is in fluid communication with manifold 104. For example, pump 42 may be connected to the one or more stopcocks of the manifold 104. In the illustrated embodiment, pump 42 is in fluid communication with manifold 104 via fluid line 107. Fluid line 107 has one end connected to and in fluid communication with pump 42 and another end opposite to the one end connected to and in fluid communication with stopcock 106a. Pump 42 may be pneumatic syringe pump, as described, for example, in U.S. Patent Publication No. 2021/0121827, which is hereby incorporated herein by reference. In operation, controlling means/controller (not shown) in the fluid distribution system causes the vacuum/pressure source of the distribution pump to operate by drawing vacuum from or pumping pressurized air to the main circuit line with designated flow rates according to the protocol.

The pump 42 may include a detection system that detects or determines the amount of fluid taken into the pump, within the pump or pushed out of the pump. For example, when the pump is a syringe, the detection system may include a position detector that detects the position of the piston, thereby detecting the amount of fluid taken into the syringe, within the syringe or pushed out of the syringe.

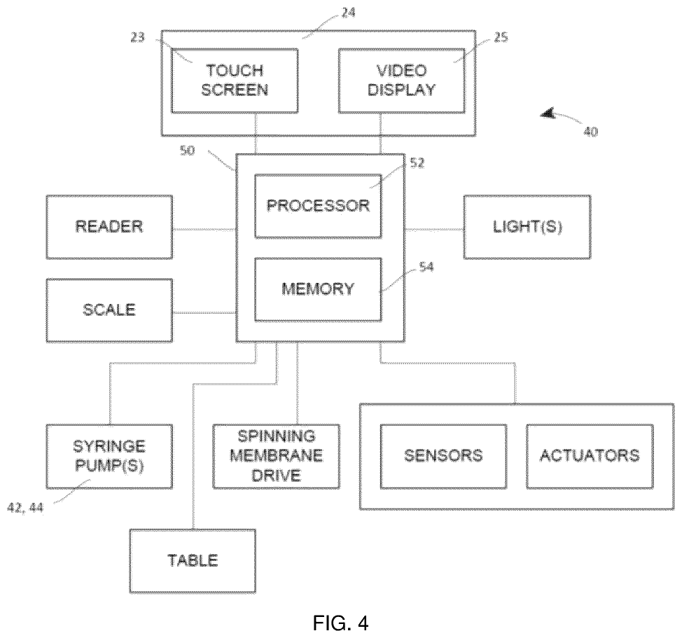

The controller 50 of the fluid distribution system 40 as illustrated in FIG. 4 includes user interface 24 which may include a touch screen 23 and a video display 25 to program the processor 52 of distribution system. The interface 24 sends signals to the controller 50.

The controller 50 may be coupled (i.e., directly, or indirectly connected) to the equipment of the system 10 and control such equipment, such as pump 42, 44, user control interface 24 and other active components as described in greater detail in U.S. Patent Publication No. 2021/0121827.

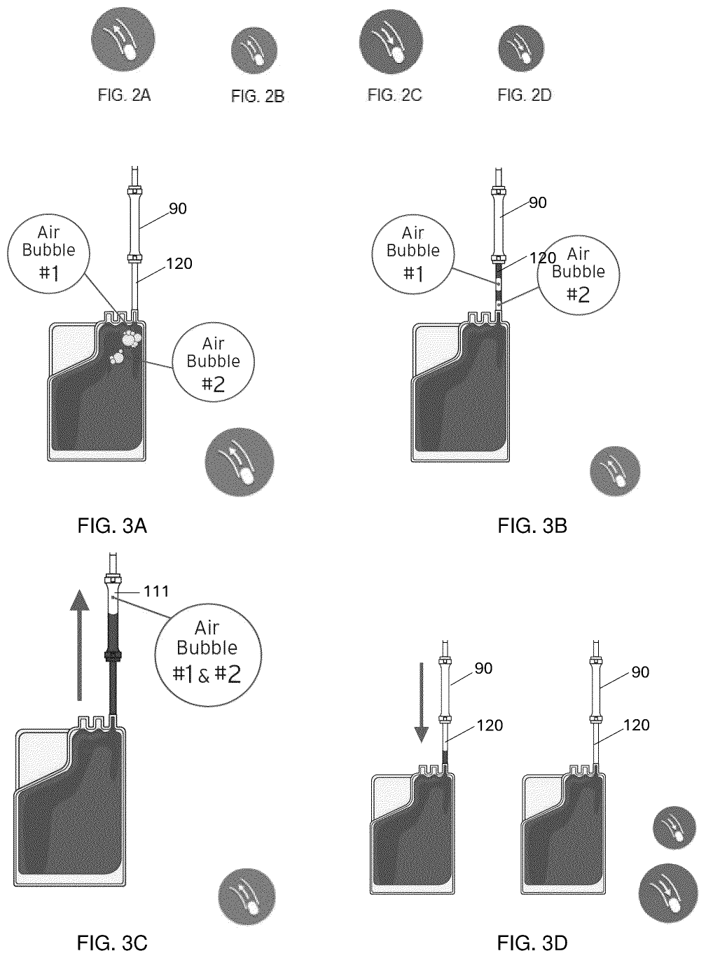

Having discussed the structure of the illustrated embodiments of the fluid circuit 100 and of the aliquoting line in detail, the method of removing gas, typically in the form of air bubbles, in the fluid flowing in the circuit 100 is now discussed with reference to FIGS. 2A-2D and 3A-3D. As much of the method of removing gas bubbles in the fluid flowing in the circuit 100 involves control of the fluid flow between the distribution system 10 and a plurality of product or aliquot containers of circuit 100, reference is also made to FIG. 1.

The controller 50 (FIG. 4) activates the distribution pump 42 to draw air through fluid lines in the fluid distribution system 10, the stopcocks 106a-e, aliquot circuit lines 102, fluid lines 107, 108a-e, 109 in the aliquot circuit 100, receptacles 90a-e and product containers 80, 82a-d, and into the distribution pump 42. At various times during the operation of the syringe pump 42 according to the selected operation mode discussed herein, the system 10 may commence with taking in air for the creation of an air chaser that may be used to push liquid through the fluid line and withdrawing air for creating a vacuum that may be used to pull liquid out from the fluid line. The amount of air taken in from the surrounding outside the system 10 and pulled out from the fluid circuit within the system 10 may be determined based on the parametric requirement designed for various pump operations as described in PCT Patent Application No. PCT/US2024/011629 and U.S. Patent Publication US 2021/0121827, which are incorporated herein by reference in their entireties.

In an exemplary system and/or method, at least four operation modes may be employed for manipulating the distribution pump 42, based on two parameters of pumping speed and direction, to treat (remove) air bubbles in the fluid circuit, and they are: Fast Pull Air Out (FIG. 2A), Slow Pull Air Out (FIG. 2B), Fast Push Air In (FIG. 2C) and Slow Push Air In (FIG. 2D). Pump speed refers to how fast the pump operates, or flow rate (e.g., liters per minute). Pump direction refers to the direction in which the pump moves the fluid, either pulls fluid in or pressures air to push fluid out. By adjusting these two parameters, the pump 42 can be set to different operation modes, control the flow rate and flow direction of the fluid.

These operation modes may involve a combination of two parameters pumping speed (fast/slow) and air pumping direction (pulls fluid in/push fluid out)—i.e., Fast Pull Air Out, Slow Pull Air Out, Fast Push Air In and Slow Push Air In. More pumping operation mode(s) may be incorporated into the system 10 depending on the nature of the application. As exemplified, the Push Air In mode designates pushing air towards the end of the main fluid flow, whereas the Pull Air Out mode designates pulling air out from the end of the main fluid flow. As further exemplified, the fast mode defines a fluid flow speed of 150 mL/min, whereas the slow mode defines a fluid flow speed of 25 mL/min. The end of the main fluid flow as embodied herein may designate product container in aliquot line.

In one exemplary method for treating air bubbles in the fluid circuit and, in particular, the aliquot fluid circuit 100 described above, after introducing fluid to the at least one product container 80, 82a-d, the method includes at least the following steps: first, placing the receptacle 90 above the product container 80, 82a-d. Next, connecting the receptacle 90 in line with the at least one fluid line 108 so that the flow passage 116 is configured in fluid communication with the at least one fluid line 108. In particular, the flow passage 116 is fluidly connected to channel 120 of the fluid line 108. Third, orienting the receptacle 90 in a default position in which the inlet 112 facing upward and the outlet 114 facing downward relative to the ground. Fourth, activating the distribution pump in a specific pump operation mode to push the fluid to the receptacle 90, and fill the product container 80 with the fluid. Then, as illustrated in FIG. 3A, activating the distribution pump 42 with a first operation mode to draw vacuum from the fluid circuit with a flow rate, as exemplified 150 mL/min, to divert a first batch of air bubbles denoted as “Air Bubble #1” in FIG. 3A in the fluid contained in the product container 80 towards the receptacle 90 until batch of residual air bubbles denoted as “Air Bubble #2” in FIG. 3A remain in the product container 80. Next, distribution pump 42 is activated in a second operation mode to draw vacuum from the fluid circuit with a flow rate as exemplified 25 mL/min, or any relatively slow flow rate as compared to the flow rate 150 mL/min in the first operation mode. This diverts a (second) batch of residual gas bubbles in the fluid contained in the product container 80 towards the receptacle 90 until first batch of air bubbles “Air Bubble #1,” the batch of residual air bubbles “Air Bubble #2” and the fluid between the air bubbles are diverted to the channel 120, as illustrated in FIG. 3B. Further, the method may include continuing to activate the distribution pump 42 in the second operation mode to draw vacuum from the fluid circuit to divert the fluid with the first batch of gas bubbles and the batch of residual gas bubbles to the receptacle 90 until the gas bubbles and the fluid surrounded the gas bubbles are diverted to the receptacle 90, as illustrated in FIG. 3C. At this point, the operator may tap the receptacle 90 to direct the gas bubbles towards the inlet 112 direction to trapping zone 111 above the fluid residing in the flow passage 116. Lastly, distribution pump 42 may be activated in a third operation mode to push the fluid collected in the receptacle 90 back to the product container 80; or activate the distribution pump 42 with a fourth operation mode to push the fluid collected in the receptacle 90 back to the product container 80. In an exemplary method, the flow rate of the first and third operation mode is faster than the flow rate of the second and fourth operation mode, respectively. In another exemplary method, the first batch of air bubbles are larger than the batch of residual gas bubbles.

In yet another exemplary method, the method of removing air bubbles includes first placing the receptacle 90 above the product container 80, 82a-d. Second, receptacle 90 is connected in line with the at least one fluid line 108 so that the flow passage 116 is configured in fluid communication with the at least one fluid line 108. In particular, the flow passage 116 is configured to be fluidly connected to channel 120 of the fluid line 108. Third, receptacle 90 may be (manually) placed in an inverted position in which the inlet 112 facing downward and the outlet 114 facing upward relative to the ground. Next, the method includes activating the distribution pump 42 with to push the fluid to fill the receptacle 90, prior to entering the product container 80. Receptacle 90 may be filled with any volume of fluid less than the device's volume capacity. Preferably, the receptacle 90 may be filled halfway so that sufficient space is provided for optimal air containment in the fluid. In this step, the air bubbles in the fluid float above the fluid residing in the receptacle 90 towards the outlet 114 direction. The method then includes orienting the receptacle 90 in the default position in which the inlet 112 facing upward and the outlet 114 facing downward relative to the ground. Flipping the receptacle 90 containing fluid with gas bubbles can help move the bubbles away from the outlet 114 as they follow the turbulence of the fluid flow in the receptacle 90. This ensures inhibiting any flow of gas (i.e., in the form of air bubbles) from the inlet 112 to the outlet 114 and thereby preventing the gas from entering the product container 80 without obstructing the fluid flow. The concept of capturing air bubbles in fluidic line relies on buoyancy, surface tension and other characteristics of bubbles in bubble-removal treatments. Bubbles are naturally buoyant and due to gas being less dense than liquid, gas bubbles will float to the gas-liquid interface, designated air trapping zone 111, where they can be captured. Next, the operator may tap the receptacle 90 to direct the air bubbles towards the inlet 112 direction to trapping zone 111 above the fluid residing in the flow passage 116. Lastly, distribution pump 42 may be activated in a third operation mode and/or a fourth operation mode to push the fluid collected in the receptacle 90 back to the product container 80.

The system may be programmable so that a user can choose a dosing protocol. The protocol may be preprogrammed or may be customized, depending on the desired use. For example, the system may include preprogrammed or saved protocols that the user may choose through the user interface. Alternatively, or in addition to the preprogrammed protocols, the system may allow the user to program in a custom protocol. The protocols may vary by amount of liquid dosed, the timing of the dosing, the order of dosing, etc.

The gas/air containment device and method as disclosed herein may be equally applicable to any fluid lines in the fluid transfer/distribution system and such application shall be within the scope of present teachings.

### Aspects

Aspect 1. An aliquot fluid circuit within a fluid distribution system having a distribution pump, the aliquot circuit includes at least one fluid line; wherein first fluid line of the at least one fluid line fluidly connected to the distribution pump; at least one or more receptacles, wherein each of the at least one or more receptacles includes a housing defining a flow passage, an inlet configured in fluid communication with the distribution pump and an outlet. The flow passage is configured to be in fluid communication between the inlet and outlet to allow the fluid flowing therethrough and being collected therein, wherein the at least one or more receptacles are configured to inhibit a flow of gas from the inlet to the outlet.

Aspect 2. The aliquot fluid circuit according to Aspect 1 further includes a manifold including at least one stopcock configured in fluid communication with said at least one fluid lines respectively, wherein the at least one stopcock fluidly connected between the at least one or more receptacles and the first fluid line and thereby in fluid communication with the distribution pump.

Aspect 3. The aliquot fluid circuit according to any one of Aspects 1 and 2 further includes at least one or more aliquot product containers, wherein each of the at least one or more aliquot product containers is fluidly connected to the at least one or more receptacles and thereby in fluid communication with the distribution pump.

Aspect 4. The aliquot fluid circuit according to Aspect 3 wherein the outlet of the at least one or more receptacle is configured in fluid communication with a respective aliquot product container.

Aspect 5. The aliquot fluid circuit according to any one of previous Aspects, wherein the fluid distribution system including a controller for controlling distribution of fluid between the fluid distribution system and the aliquot fluid circuit.

Aspect 6. The aliquot fluid circuit according to any one of previous Aspects, wherein the controller activates the distribution pump in at least one operation mode, and wherein the at least one operation mode is parameterized by flow rate and pump direction.

Aspect 7. The aliquot fluid circuit according to Aspect 6, wherein the flow rate is 150 mL/min.

Aspect 8. The aliquot fluid circuit according to Aspect 6, wherein the flow rate is 25 mL/min.

Aspect 9. The aliquot fluid circuit according to Aspect 6, wherein the pump direction comprising first direction and second direction; wherein the second direction is opposite to the first direction.

Aspect 10. The aliquot fluid circuit according to Aspect 9, wherein the distribution pump operating in the first direction pumps pressurized air to the aliquot fluid circuit.

Aspect 11. The aliquot fluid circuit according to Aspect 9, wherein the distribution pump operating in the second direction draws vacuum from the aliquot fluid circuit.

Aspect 12. The aliquot fluid circuit according to any one of previous Aspects, wherein the fluid circuit includes a vent.

Aspect 13. The aliquot fluid circuit according to any one of Aspects 2-12, further including a latch for holding the manifold.

Aspect 14. A method for treating gas bubbles in an aliquot fluid circuit according to any one of Aspects 1-13 upon filing fluid to the at least one or more product containers, wherein the method comprising steps: (i) placing the receptacle above the product container; (ii) connecting the receptacle in line with the at least one fluid line; wherein the flow passage configured in fluid communication with the at least one fluid line; (iii) orienting the receptacle in a default position in which the inlet facing upward and the outlet facing downward relative to the ground; (iv) activating the distribution pump to push the fluid to the receptacle, and fill the product container with the fluid; (v) activating the distribution pump with a first or second operation mode to draw vacuum from the fluid circuit to divert the gas bubbles residing in the fluid contained in the product container towards the receptacle; (vi) tapping the receptacle to direct the gas bubbles towards the inlet direction to trapping zone above the fluid residing in the flow passage; (vii) activating the distribution pump in a third operation mode to push the fluid in the receptacle into the product container; and/or (viii) activating the distribution pump in a fourth operation mode to push the fluid in the receptacle into the product container.

Aspect 15. The method according to Aspect 14, wherein the flow rate of the first and third operation mode is faster than the flow rate of the second and fourth operation mode, respectively.

Aspect 16. The method according to Aspect 14 or 15, wherein the first and second operation modes operate the distribution pump in the first direction.

Aspect 17. The method according to Aspect 14 or 15, wherein the third and fourth operation modes operate the distribution pump in the second direction.

Aspect 18. The method according to Aspect 15, wherein the flow rate of the first and third operation modes is 150 mL/min.

Aspect 19. The method according to Aspect 15, wherein the flow rate of the second and fourth operation modes is 25 mL/min.

Aspect 20. A method for treating gas bubbles in the aliquot fluid circuit according to any one of Aspects 1-13 upon filling fluid to the at least one or more product containers, the method comprising steps: (i) placing the receptacle above the product container; (ii) connecting the receptacle in line with the at least one fluid line; wherein the flow passage of the receptacle configured in fluid communication with the at least one fluid line; (iii) orienting the receptacle in an inverted position in which the inlet facing downward and the outlet facing upward relative to the ground; (iv) activating the distribution pump to push the fluid to fill the receptacle, prior to entering the product container; wherein the gas bubbles float above the fluid residing in the receptacle towards the outlet direction; (v) orienting the receptacle in the default position in which the inlet facing upward and the outlet facing downward relative to the ground to inhibit the gas bubbles flow from the inlet to the outlet without obstructing the fluid flow; (vi) tapping the receptacle to direct the gas bubbles towards the inlet direction to trapping zone above the fluid residing in the flow passage; (vii) activating the distribution pump with a third operation mode to push the fluid in the receptacle into the product container; or (viii) activating the distribution pump with a fourth operation mode to push the fluid in the receptacle into the product container.

Aspect 21. The method according to Aspect 20, wherein filling halfway of the receptacle with the fluid in step (iv).

Aspect 22. The method according to Aspect 20, wherein the flow rate of the first and third operation mode is faster than the flow rate of the second and fourth operation mode, respectively.

Aspect 23. The method according to Aspect 20 or 22, wherein the first and second operation modes operate the distribution pump in the first direction.

Aspect 24. The method according to claim 20 or 22, wherein the third and fourth operation modes operates the distribution pump in the second direction.

In the foregoing specification, specific embodiments have been described. However, one of ordinary skills in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings.

1. An aliquot fluid circuit within a fluid distribution system having a distribution pump, the aliquot circuit comprising:
at least one fluid line; wherein first fluid line of the at least one fluid line is fluidly connected to the distribution pump;
at least one or more receptacles, wherein each of the at least one or more receptacles comprises a housing defining a flow passage, an inlet configured in fluid communication with the distribution pump and an outlet; the flow passage configured in fluid communication between the inlet and outlet to allow the fluid flowing therethrough and being collected therein;
wherein the at least one or more receptacles are configured to inhibit a flow of gas from the inlet to the outlet.

2. The aliquot fluid circuit according to claim 1 further comprising a manifold including at least one stopcock configured in fluid communication with said at least one fluid lines respectively, wherein the at least one stopcock fluidly connected between the at least one or more receptacles and the first fluid line and thereby in fluid communication with the distribution pump.

3. The aliquot fluid circuit according to claim 1 further comprising at least one or more aliquot product containers, wherein each of the at least one or more aliquot product containers is fluidly connected to the at least one or more receptacles and thereby in fluid communication with the distribution pump.

4. The aliquot fluid circuit according to claim 3 wherein the outlet of the at least one or more receptacle is configured in fluid communication with a respective aliquot product container.

5. The aliquot fluid circuit according to claim 1, wherein the fluid distribution system including a controller for controlling distribution of fluid between the fluid distribution system and the aliquot fluid circuit.

6. The aliquot fluid circuit according to claim 1, wherein the controller activates the distribution pump in the at least one operation mode, and wherein the at least one operation mode is parameterized by flow rate and pump direction.

7. The aliquot fluid circuit according to claim 6, wherein the flow rate is 150 mL/min.

8. The aliquot fluid circuit according to claim 6, wherein the flow rate is 25 mL/min.

9. The aliquot fluid circuit 100 according to claim 6, wherein the pump direction comprising first direction and second direction; wherein the second direction is opposite to the first direction.

10. The aliquot fluid circuit according to claim 9, wherein the distribution pump operating in the first direction pumps pressurized air to the aliquot fluid circuit.

11. The aliquot fluid circuit according to claim 9, wherein the distribution pump operating in the second direction draws vacuum from the aliquot fluid circuit.

12. The aliquot fluid circuit according to claim 1, wherein the circuit includes a vent.

13. The aliquot fluid circuit according to claim 2, further including a latch for holding the manifold.

14. A method for treating gas bubbles in the aliquot fluid circuit according to claim 1 upon filling fluid to the at least one or more product containers, wherein the method comprising steps:
(i) placing the receptacle above the product container;
(ii) connecting the receptacle in line with the at least one fluid line; wherein the flow passage configured in fluid communication with the at least one fluid line;
(iii) orienting the receptacle in a default position in which the inlet facing upward and the outlet facing downward relative to the ground;
(iv) activating the distribution pump to push the fluid to the receptacle 90, and fill the product container with the fluid;
(v) activating the distribution pump in a first or second operation mode to draw vacuum from the fluid circuit to divert the gas bubbles residing in the fluid contained in the product container towards the receptacle;
(vi) tapping the receptacle to direct the gas bubbles towards the inlet direction to a trapping zone above the fluid residing in the flow passage;
(vii) activating the distribution pump in a third operation mode to push the fluid in the receptacle into the product container; or
(viii) activating the distribution pump with a fourth operation mode to push the fluid in the receptacle into the product container.

15. The method according to claim 14, wherein the flow rate of the first and third operation mode is faster than the flow rate of the second and fourth operation mode, respectively.

16. The method according to claim 14, wherein the first and second operation modes operate the distribution pump in the first direction.

17. The method according to claim 14, wherein the third and fourth operation modes operates the distribution pump in the second direction.

18. The method according to claim 15, wherein the flow rate of the first and third operation modes is 150 mL/min.

19. The method according to claim 15, wherein the flow rate of the second and fourth operation modes is 25 mL/min.

20. A method for treating gas bubbles in the aliquot fluid circuit according to claim 1 upon filling fluid to the at least one or more product containers, the method comprising steps:
(i) placing the receptacle above the product container;
(ii) connecting the receptacle in line with the at least one fluid line; wherein the flow passage of the receptacle configured in fluid communication with the at least one fluid line;
(iii) orienting the receptacle in an inverted position in which the inlet facing downward and the outlet facing upward relative to the ground;
(iv) activating the distribution pump with to push the fluid to fill the receptacle, prior to entering the product container; wherein the gas bubbles float above the fluid residing in the receptacle towards the outlet direction;
(v) orienting the receptacle in the default position in which the inlet facing upward and the outlet facing downward relative to the ground to inhibit the gas bubbles flow from the inlet to the outlet without obstructing the fluid flow;
(vi) tapping the receptacle to direct the gas bubbles towards the inlet direction to trapping zone above the fluid residing in the flow passage;
(vii) activating the distribution pump with a third operation mode to push the fluid in the receptacle into the product container; or
(viii) activating the distribution pump with a fourth operation mode to push the fluid in the fluidic device into the product container.

21. The method according to claim 20, wherein filling halfway of the receptacle with the fluid in step (iv).

22. The method according to claim 20, wherein the flow rate of the first and third operation mode is faster than the flow rate of the second and fourth operation mode, respectively.

23. The method according to claim 20, wherein the first and second operation modes operate the distribution pump in the first direction.

24. The method according to claim 20, wherein the third and fourth operation modes operates the distribution pump in the second direction.
